(19) United States
(12) Patent Application Publication
(10) Pub. No.: US 2025/0099531 A1
(43) Pub. Date: Mar. 27, 2025

(54) SYNERGISTIC COMPOSITIONS FOR ENHANCING TFEB-MEDIATED INTRACELLULAR CLEARANCE

(71) Applicant: CELAGENEX RESEARCH (INDIA) PVT. LTD., Thane West (IN)

(72) Inventors: Rajaram SAMANT, Thane West (IN); Rajendra Prasad T., Jaipur (IN)

(73) Assignee: CELAGENEX RESEARCH (INDIA) PVT. LTD., Thane West (IN)

(21) Appl. No.: 18/832,266

(22) PCT Filed: Jan. 20, 2023
(86) PCT No.: PCT/IN2023/050063

§ 371 Date: Jul. 23, 2024

(30) Foreign Application Priority Data
Jan. 23, 2022 (IN) 202121038110

(51) Int. Cl. A61K 38/05; A61K 31/132; A61P 43/00

(52) U.S. Cl. CPC A61K 38/05; A61K 31/132; A61P 43/00

(57) ABSTRACT

Compositions for enhancing TFEB-mediated intracellular clearance are herein. Particularly, a complex comprising synergistic combination of polyamine and histidine-containing dipeptide present in a specific weight ratio along with pharmaceutically acceptable excipients is provided herein. More particularly, the composition comprises a synergistic blend of N-(3-Aminopropyl)-1,4-butanediamine and N-α-Acetyl-N-β-alanyl-L-histidine and salts thereof along with pharmaceutically acceptable excipients. Furthermore, the present synergistic composition is useful for improving or inducing autophagy, lysosomal biogenesis, mitophagy and lipophagy.

### FIELD OF THE INVENTION

The present invention relates to compositions for enhancing TFEB-mediated intracellular clearance. Particularly, the invention relates to a complex comprising synergistic combination of polyamine and histidine-containing dipeptide present in a specific weight ratio along with pharmaceutically acceptable excipients. More particularly, the invention relates to compositions comprising blend of N-(3-Aminopropyl)-1,4-butanediamine and N-α-Acetyl-N-β-alanyl-L-histidine and salts thereof along with pharmaceutically acceptable excipients. Further the present synergistic composition is useful for improving autophagy, lysosomal biogenesis, mitophagy and lipophagy.

### BACKGROUND OF THE INVENTION

Generation of new cells and subsequent destruction of dead cells is necessary for normal cellular homeostasis, wound healing, and immune responses in human. The ruin left behind in its wake would be catastrophic, if not for the efficient work of the phagocytic system. Despite the constant, homeostatic turnover of cells, as well as cell death induced by stress, damage, or infection, it is rare to observe dead cells (either programmed or traumatic cell death) under normal physiological conditions. The average adult human loses between 50 to 70 billion cells each day due to apoptosis, and a child between the ages of 8 to 14 loses approximately 20-30 billion cells per day due to cell death.

In addition to apoptosis, a number of regulated cell death modalities have come to light in the recent years. Among these, those that occur during development and homoeostasis include apoptosis, necroptosis (also called programmed necrosis) and autophagy-dependent cell death. The prompt clearance of dead cells involves molecular steps that include the recruitment of phagocytes towards apoptotic cells through ‘find-me’ signals and the recognition of ‘eat-me’ signals on apoptotic cells that trigger engulfment.

Defects in dead cell removal are associated with the initiation and progression of a number of pathological conditions, including inflammation and autoimmunity. The process of dead cell clearance can be manipulated potentially by pharmacological means to treat a variety of human diseases. Dead cell clearance can be done by different mechanism which includes autophagy, mitophagy and lipophagy.

Further cell senescence is increasingly recognized as a major contributor to the loss of health and fitness associated with ageing. Senescent cells accumulate dysfunctional mitochondria, oxidative phosphorylation efficiency is reduced, and the reactive oxygen species production is increased. By removing damaged macromolecules or organelles, autophagy prevents garbage catastrophe, thus exerting an anti-senescence role. Macro, micro, and chaperone mediated autophagy are mediated by autophagy-related genes and their associated enzymes. Macro autophagy is further divided into bulk and selective autophagy. The selective autophagy is the autophagy of organelles comprising mitochondria (mitophagy), endoplasmic reticulum (reticulophagy), microorganisms (xenophagy) and aggregated proteins (aggrephagy), lipid droplets (lipophagy), perioxisomes (pexophagy), ribosomes (ribophagy) and like thereof.

Autophagy, lipophagy and mitophagy are important cellular processes that are responsible for breaking down cellular contents, preserving energy and safeguarding against accumulation of damaged and aggregated biomolecules. Autophagy is an essential intracellular clearance mechanism, which helps to maintain normal cell homeostasis and it is also a critical regulator of the intracellular immune system. Autophagy dysfunction has been associated with a variety of diseases such as cancer, muscular disorders, pathogenic infections, cerebral ischemia, neurodegeneration, psychiatric disorders, neurodegenerative diseases like [Alzheimer's disease (AD), Parkinson's disease (PD), and Huntington's disease (HD)].

Further, autophagy is generally considered as a cell survival/protection mechanism because it removes toxic or obsolete proteins and organelles; and recycles the degradation products for use as sources for energy and metabolites in anabolic pathways. Notably transcription factor EB (TFEB), a basic helix-loop-helix-leucine-zipper (bHLH-Zip) protein in the microphthalmia/transcription factor E (MiT/TFE) family, primarily controls the expression of genes in the autophagy-lysosomal pathway. Transcription factor acts as a master regulator of lysosomal biogenesis, autophagy, lysosomal exocytosis, lipid catabolism, energy metabolism and immune response. TFEB regulates autophagic flux by promoting the biogenesis of lysosomes, formation of autophagosomes, and fusion with lysosomes, thereby facilitating substrate clearance. TFEB also functions in selective autophagy and lysosomal exocytosis.

Interestingly, overexpression of TFEB enhances the degradation of bulk amounts of substrates, lipid droplets, and damaged mitochondria and alleviates the phenotypes associated with various diseases. Moreover, TFEB is involved in intracellular clearance by enhancing lysosomal biogenesis and function, autophagy, and lysosomal exocytosis. TFEB overexpression improves the rate of degradation of the cargo, e.g., long-lived proteins and enhances the elimination of lipid droplets and damaged mitochondria. Hence, TFEB is the central modulator of the autophagy-lysosomal pathway and organelle-specific autophagy, including lipophagy and mitophagy. [Cell Death Discovery volume 6, Article number: 32 (2020)].

The dissection of the cellular processes controlled by TFEB may have relevance for the therapy of various diseases, including autophagy and vesicle trafficking in addition to specific degradative pathways. The factors affecting or striking TFEB become needful for establishing therapeutic approach of TFEB-mediated intracellular clearance.

The polyamines are involved in various biological processes, notably in cell proliferation and differentiation, and have antioxidant properties. Dietary polyamines have important implications in human health, mainly in the intestinal maturation and in the differentiation and development of immune system. In addition to endogenous synthesis, food is an important source of polyamines. Polyamines can be found in all types of foods in a wide range of concentrations. The main polyamine in plant-based products is spermidine, however the spermine content is generally higher in animal-derived foods. [Munõz-Esparza et al. (2019) Polyamines in Food. Front. Nutr. 6:108].

A decline in polyamine levels with age has been associated with the pathophysiology of ageing. Among the other amino acids, hypusine is an amino acid that appears in only a single protein i.e., eIF5A. Declining levels of polyamines result in a loss of functional hypusinated eIF5A, due to which TFEB is not efficiently translated, and autophagy genes are therefore not efficiently transcribed. Restoring polyamine levels reverses this sequence and B cell function is restored. Hypusine is generated by a two-step enzymatic process that transfers an n-butylamine moiety from spermidine to the F-amino group of a specific lysine side chain in the eIF5A polypeptide. [Mol Cell, 2019 Oct. 3; 76(1):5-7].

‘Polyamine formed from putrescine is an endogenous polyamine metabolite which provokes autophagy in vivo and revitalizes memory B cell responses. N-(3-Aminopropyl)-1,4-butanediamine post-translationally modifies the translation factor eIF5A, which is essential for the synthesis of the autophagy transcription factor TFEB. N-(3-Aminopropyl)-1,4-butanediamine is depleted in the elderly, leading to reduced TFEB expression and autophagy. [Zhang et al., 2019, Molecular Cell 76, 110-125]. Moreover, N-(3-Aminopropyl)-1,4-butanediamine supplementation restores cellular pathway and improves the responses of old human B cells.

WO2019/121856A1 discloses compositions of an autophagy inducer and high protein for induction of autophagy in a patient in the Intensive Care Unit (ICU), and/or an ageing patient, or an individual with sarcopenia.

WO2018/087388 A1 relates to (N-(3-Aminopropyl)-1,4-diaminobutane) is a polycationic aliphatic amine which plays several roles in cell survival.

Recent research shows that N-(3-Aminopropyl)-1,4-butanediamine can act via other mechanisms, namely inflammation reduction, lipid metabolism and regulation of cell growth, proliferation, and death. [Gerontology 2014; 60:319-326]. It is evident that polyamine based supplement, particularly N-(3-Aminopropyl)-1,4-butanediamine gives better autophagy regulatory mechanism mediated by eIF5A at the translational level.

The inventors of the present invention have found that N-(3-Aminopropyl)-1,4-butanediamine is a therapeutically active candidate for intracellular clearance as it post-translationally modifies the translation factor eIF5A, which is essential for the synthesis of the autophagy transcription factor TFEB. However, a single dose of N-(3-Aminopropyl)-1,4-butanediamine may not be much effective for the intracellular clearance. Therefore, there is a need to find out other ingredients that synergistically increase and ameliorate TFEB gene expression.

Recent studies have shown that the sub-cellular localization and activity of TFEB are regulated by mechanistic target of rapamycin (mTOR)-mediated phosphorylation. Additionally, mechanistic target of rapamycin complex 1 (mTORC1) is an atypical protein kinase that is stimulated by the nutrient content and provides balance between anabolism and catabolism. Ser211 is phosphorylated by mTORC1, which phosphorylates TFEB at Ser211 and induces the binding between TFEB and 14-3-3 proteins, thereby inhibiting TFEB from shuttling into the nucleus. After mTORC1 is inhibited, TFEB gets dephosphorylated and is translocated into the nucleus. Moreover, N-(3-Aminopropyl)-1,4-butanediamine and resveratrol did not affect the phosphorylation of mechanistic target of rapamycin (mTOR) nor that of its substrate ribosomal protein kinase, which suggests that resveratrol and N-(3-Aminopropyl)-1,4-butanediamine induce autophagy through AMP-dependent kinase/mTOR-independent convergent pathways.

Current mTOR inhibitors, such as rapamycin and torin1, activate TFEB by promoting TFEB nuclear translocation. However, their pharmacokinetic profile and side effects make them less likely to be useful for long-term use in patients with neurodegenerative diseases. Disaccharides, such as trehalose and sucrose, activate TFEB in an mTOR-independent manner and may be beneficial for neurodegenerative diseases. However, the blood-brain barrier (BBB) permeability of trehalose and sucrose is poor.

Some patent and non-patent publications provide compounds that directly bind to and activate TFEB without inhibiting mTOR pathway, thus eliminating possible mTOR-associated complications.

Particularly, U.S. Pat. No. 9,351,946B2 relates to a composition comprising an autophagy enhancement compound i.e., monocarbonyl analogue of curcumin. However, the bioavailability of curcumin is frequently a challenging factor. Usually, formulation of curcumin needs hydrophilic carrier, cellulosic derivatives, and natural antioxidants to increase curcuminoid appearance in the blood.

Furthermore, it is well evident that inhibition of mTORC1 strongly induces autophagy by dephosphorylating TFEB translocated to the nucleus to induce the transcription of target genes. Thus, a cytosol-to-nucleus signalling pathway regulates cellular energy metabolism through TFEB. [J Cell Sci. 2016 Jul. 1; 129(13): 2475-2481].

It is well established that inhibition of the mammalian target of rapamycin (mTOR) pathway plays a key role in autophagy. Carnosine has a profound and significant effect on autophagy. Treatment with carnosine significantly attenuated autophagic signalling in the ischemic brain, with improvement of brain mitochondrial function and mitophagy signalling. The protective effect of carnosine against autophagy was also confirmed in primary cortical neurons. [Stroke 2014 August; 45(8):2438-2443].

Carnosine (0-alanyl-L-histidine), a naturally occurring dipeptide consisting of 0-alanine and histidine, is synthesized by carnosine synthetase. Moreover, the dipeptide, similar with rapamycin, inhibits the activation of Akt, mTOR and p70S6K protein. [J Cancer. 2014; 5(5): 382-389].

Another finding revealed that carnosine prevents osteogenic differentiation of Vascular smooth muscle cells (VSMCs) by suppressing mTOR signalling and regulating increased production of reactive oxygen species (ROS). [Aging Medicine. 2020; 3:153-158].

Unfortunately, there exist questions about the efficacy of oral supplementation with carnosine. Most of the carnosine absorbed from the gut is destroyed in the bloodstream by enzymes called carnosinases. These enzymes readily split carnosine back into its two constituent amino acids, alanine and histidine. This fact has led to trials of intranasal administration of carnosine.

Surprisingly, the inventors of the present invention have found that the N-α-Acetyl-N-β-alanyl-L-histidine is a better mTOR inhibitor as compared to carnosine. As a supplement, N-α-Acetyl-N-β-alanyl-L-histidine improve energy levels. It has an additional acetyl group bound to L-carnosine, where the acetyl group protects the L-carnosine, keeping it from being degraded. It means that N-α-Acetyl-N-β-alanyl-L-histidine is more effective than taking L-carnosine directly. It also makes it more efficient, so that it can take a lower dose when supplementing. Although the body produces L-carnosine naturally, N-α-Acetyl-N-β-alanyl-L-histidine supplements can increase its levels.

In view of the aforementioned, the inventors of the present invention have done rigorous experiments and developed cost-effective, non-toxic, safe, and therapeutically active combination of N-(3-Aminopropyl)-1,4-butanediamine and its bioavailable salt along with N-α-Acetyl-N-β-alanyl-L-histidine (NAC) in a specific ratio, where both the active moieties work synergistically to enhance TFEB-mediated intracellular clearance.

### OBJECTIVE OF THE INVENTION

The primary objective of the invention is to provide synergistic compositions for maintaining normal cell homeostasis.

Another objective of the invention is to provide a complex that harnesses to reverse immune senescence in humans by targeting TFEB activity.

Another objective of the invention is to provide polyamine-based medicinal composition that give better autophagy regulatory mechanism mediated by eIF5A at the translational level.

Further objective of the invention is to provide composition that facilitates intracellular degradation and improves mitochondrial function and cell health.

Another objective of the invention is to provide bioavailable, safe, non-toxic and cost-effective composition.

Yet another objective of the invention is to provide synergistic polyamine-based remedy for cellular recycling with no side effects.

### SUMMARY OF THE INVENTION

To meet the above objectives, the inventors of the present invention performed thorough experiments to establish the significant effect of the active ingredients or minerals or chemicals or polyamines or peptide or nutrients present in the composition for enhancing TFEB-mediated intracellular clearance in a subject in need thereof in safer way.

In an aspect, the invention relates to synergistic compositions comprising therapeutically active nutrients along with pharmaceutically acceptable carriers for improving autophagy, lysosomal biogenesis, mitophagy and lipophagy.

In another aspect, the invention relates to synergistic compositions comprising exogenous combination of ingredients, present in a specific weight ratio to ameliorate TFEB gene expression. TFEB positively regulates the transcription of genes involved in all steps of lysosome biogenesis. TFEB promotes lysosomal proliferation, acidification, and exocytosis, and induces genes involved in autophagic pathways. Together, TFEB-modulated processes promote clearance of lysosomal and autophagic substrate.

In a particular aspect, the present invention provides synergistic composition for inducing cellular degradation of damaged and aged cells, comprising specific combination of N-(3-Aminopropyl)-1,4-butanediamine and N-α-Acetyl-N-β-alanyl-L-histidine and salts thereof along with pharmaceutically acceptable excipients.

In another aspect, the present invention provides synergistic compositions for treatment of depression disorder comprising administration of effective dose of N-(3-Aminopropyl)-1,4-butanediamine along with N-α-Acetyl-N-β-alanyl-L-histidine (NAC) or salts thereof, wherein N-(3-Aminopropyl)-1,4-butanediamine enhances hypusinated translation factor eIF5A and the NAC simultaneously inhibits the activation of the mammalian target of rapamycin (mTOR) pathway, both synergistically promoting efficient shuttling of TFEB from cytosol to the nucleus and improving transcription of multiple genes implicated in the formation of autophagosomes and lysosomes.

In yet another aspect, the invention relates to synergistic compositions comprising exogenous combination of N-(3-Aminopropyl)-1,4-butanediamine and salts thereof, which are present in a range of 0.1-500 mg and N-α-Acetyl-N-β-alanyl-L-histidine and salts thereof, which are present in a range of 0.1-350 mg along with pharmaceutically acceptable excipients/carriers.

In yet one more aspect, the invention discloses synergistic composition useful for improving autophagy, lysosomal biogenesis, mitophagy and lipophagy. Particularly the composition provides therapeutic approach in the treatment of neurodegenerative diseases, (including Alzheimer's disease, Parkinson's disease, and Huntington's disease), cardiomyopathy, diabetes, liver diseases, muscle diseases, cardiac diseases, cancer, age-related disorders, infection, immunity, autoimmune and inflammatory diseases, pregnancy complications, foetal and infant brain development.

In addition to elimination of intracellular aggregates and damaged organelles, the present composition reverses immune senescence, protects against genome instability, organismal ageing, prevents necrosis and reduces cellular reactive oxygen species (ROS) production.

### BRIEF DESCRIPTION OF DRAWINGS

FIG. 1 illustrates schematic representation of synergistic effect of N-(3-Aminopropyl)-1,4-butanediamine and NAC for promoting efficient shuttling of TFEB.

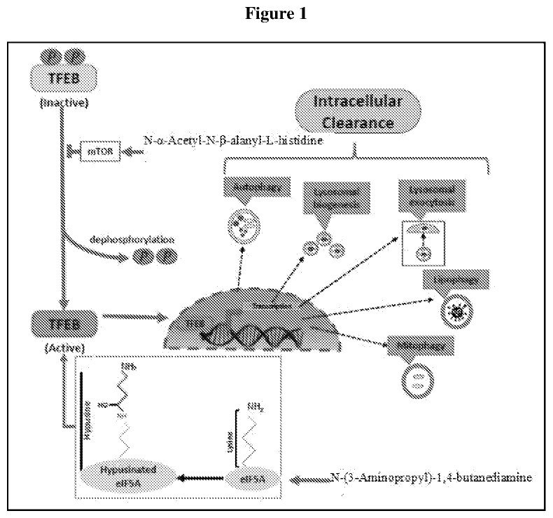

FIG. 2 illustrates Percentage of THP-1 cells in autophagy analysed by CYTO-ID where G1—Cell Control (untreated); G2—Rapamycin (Reference Standard); G3—Test-1: N-(3-Aminopropyl)-1,4 butanediamine—3 mg; G4—Test-2: N-α-Acetyl-N-β-alanyl-L-histidine-1 mg; G5—Test 3: N-(3-Aminopropyl)-1,4 butanediamine+N-α-Acetyl-N-β-alanyl-L-histidine (3 mg+0.5 mg); G6—Test 4: N-(3-Aminopropyl)-1,4 butanediamine+N-α-Acetyl-N-β-alanyl-L-histidine (3 mg+1 mg); G7—Test-5: N-(3-Aminopropyl)-1,4 butanediamine+N-α-Acetyl-N-β-alanyl-L-histidine (0.1 mg+5 mg).

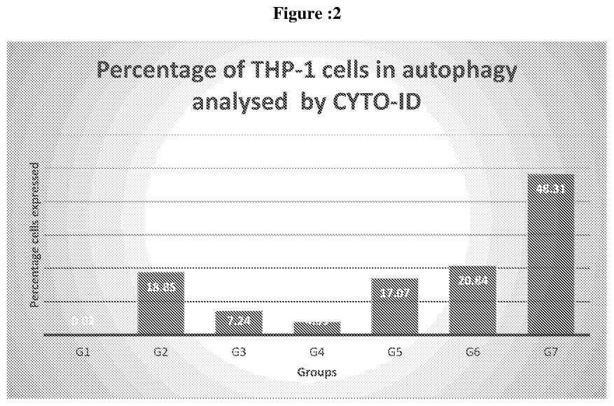

### DETAILED DESCRIPTION OF THE INVENTION

The invention will now be described in detail in connection with certain preferred and optional embodiments, so that various aspects thereof may be more fully interpreted and comprehended. However, any skilled person or artisan will appreciate the extent to which such embodiments could be generalized in practice.

It is further to be understood that all terminology used herein is for the purpose of describing particular embodiments only and is not intended to be limiting in any manner or scope. Unless defined otherwise, all technical and scientific expressions used herein have the same meaning as commonly understood by one of ordinary skill in the art to which embodiments of the invention pertain. In describing and claiming the embodiments of the present invention, the following terminology can be used in accordance with the definitions set out below which are known in the state of art.

The singular forms “a,” “an,” and “the” include plural reference unless the context clearly dictates otherwise. Also, the term “composition” does not limit the scope of the invention for multiple compositions that can be illustrated for best mode of the invention.

The term “pharmaceutically/nutraceutically acceptable salt,” as used herein, represents those salts which are within the scope of sound medical judgment and suitable for use in contact with the tissues of humans and animals without undue toxicity, irritation, allergic response and the like and are commensurate with a reasonable benefit/risk ratio. Particularly, the term “pharmaceutically-acceptable salts” refer to relatively non-toxic, inorganic and organic acid addition salts of compounds, amino acid salts, sugar-based salts, alkali or alkaline earth metal salts, as well as solvates, co-crystals, polymorphs and the like of the salts.

All modifications and substitutions that come within the meaning of the description and the range of their legal equivalents are to be embraced within their scope. A description using the transition “comprising” allows the inclusion of other elements to be within the scope of the invention.

In a preferred embodiment, the invention provides novel and potent synergistic compositions for enhancing TFEB-mediated intracellular clearance.

In another embodiment, the invention relates to complex comprising synergistic combination of polyamine and histidine-containing dipeptide present in a specific weight ratio along with pharmaceutically acceptable excipients.

In particular embodiment, the invention provides composition comprising synergistic exogenous blend of N-(3-Aminopropyl)-1,4-butanediamine and N-α-Acetyl-N-β-alanyl-L-histidine and salts thereof along with pharmaceutically acceptable excipients, wherein the N-(3-Aminopropyl)-1,4-butanediamine and N-α-Acetyl-N-β-alanyl-L-histidine are present in a specific weight ratio. This biologically active composition is composed of synergistic combination of N-(3-Aminopropyl)-1,4-butanediamine and N-α-Acetyl-N-β-alanyl-L-histidine which are present in a therapeutically effective amount. The composition significantly promotes TFEB-mediated intracellular clearance or degradation. Particularly, the synergistic combination of N-(3-Aminopropyl)-1,4-butanediamine and NAC enhancing TFEB-mediated protein degradation and organelle turnover is required for the survival of cells. Furthermore, the composition improves mitochondrial function and cell health with enhanced bioavailability, solubility, and therapeutic efficacy.

In another embodiment, the invention provides synergistic compositions to maintain cellular homeostasis and functions and plays an important role in the survival of cellular organisms.

In another embodiment, the invention provides synergistic compositions for inducing or promoting autophagy comprising exogenous blend or combination of effective amount of N-(3-Aminopropyl)-1,4-butanediamine and NAC or salts thereof along with pharmaceutically acceptable excipients.

N-(3-Aminopropyl)-1,4-butanediamine is a polyamine compound found in ribosomes and living tissues and having various metabolic functions within organisms. N-(3-Aminopropyl)-1,4-butanediamine [N-(3-aminopropyl)-1,4-diaminobutane] is a saturated linear triamine often classified as an intermediate in the synthesis of Carnosine. N-(3-Aminopropyl)-1,4-butanediamine is a precursor of spermine. N-(3-Aminopropyl)-1,4-butanediamine is a polyamine formed from putrescine. It has molecular formula C₇H₁₉N₃ and molar mass 145.25 g/mol and is a small, water-soluble, amphiphilic molecule with the ability to penetrate biological membranes. One of the best-known grain dietary sources is wheat germ, containing as much as 243 mg/kg. The N-(3-Aminopropyl)-1,4-butanediamine rich wheat germ extract is GRAS listed nutrient.

Particularly N-(3-Aminopropyl)-1,4-butanediamine promotes the hypusination of EIF5A, which regulates the translation of TFEB, which in turn regulates autophagosomal and lysosomal biogenesis. N-(3-Aminopropyl)-1,4-butanediamine can reverse the age-dependent decline of the hypusinated EIF5A-TFEB-autophagy axis and restore B cell function. N-(3-Aminopropyl)-1,4-butanediamine is an essential substrate of the hypusination process, in which it donates an aminobutyl moiety to a specific lysine on EIF5A (eukaryotic translation initiation factor 5A) to form hypusine. EIF5A is a translation factor with well-defined function of facilitating the elongation step during protein synthesis involving its hypusine residue. The hypusinated EIF5A thus activates rapid TFEB translocation to the nucleus and activates the transcription of its target genes.

In yet another embodiment, the present invention provides a synergistic composition comprising a therapeutically effective amount of N-(3-Aminopropyl)-1,4-butanediamine or pharmaceutically acceptable salts thereof, wherein N-(3-Aminopropyl)-1,4-butanediamine is present in a range of 1-200 mg of the total composition.

N-acetyl-β-alanyl-L-histidine or N—(N-Acetyl-β-alanyl) histidine (abbreviated NAC) is a dipeptide. NAC is crystalline solid chemically known as (S)-2-(3-Acetylamino propionyl amino)-3-(3H-imidazol-4-yl) propionic acid with molecular formula C₁₁H₁₆N₄O₄ and formula weight 268.27 g/mol.

The NAC molecular structure is identical to carnosine with the exception that it carries an additional acetyl group. The acetylation makes NAC more resistant to degradation by carnosinase, an enzyme that breaks down carnosine to its constituent amino acids, beta-alanine and histidine. N-α-Acetyl-N-β-alanyl-L-histidine is one of the best ways to increase L-carnosine levels in the body. Its effects are similar to what you get from taking L-carnosine, as it converts to L-carnosine in the body. However, it also prevents degradation of the L-carnosine, so that human body gets more of the molecule, leading to a more significant therapeutic effect.

Under normal nutrient conditions, TFEB is phosphorylated in Ser211 in an mTORC1-dependent manner. This phosphorylation promotes association of TFEB with members of the YWHA (14-3-3) family of proteins and retention of the transcription factor in the cytosol. Pharmacological or genetic inhibition of mTORC1 causes dissociation of the TFEB/YWHA complex and rapid transport of TFEB to the nucleus where it increases transcription of multiple genes implicated in autophagy and lysosomal function.

In one embodiment the invention provides synergistic composition wherein the NAC acts as mTOR inhibitor that helps in the maintenance of cellular homeostasis by regulating autophagy at the transcriptional level.

Particularly, the administration of effective amount of NAC promotes dephosphorylation of TFEB proteins complex thus makes phosphate group free and activates TFEB for effective shuttling from cytosol to the nucleus for transcription.

In another embodiment, the synergistic composition comprises a therapeutically effective amount of NAC present in a range of 1-1000 mg of the total composition. In yet another embodiment, NAC is present in a range of 1-500 mg of the total composition. The term “therapeutically effective amount” denotes an amount that reduces the risk, potential, possibility or occurrence of a disease or disorder, or provides advanced alleviation, mitigation, and/or reduction, restoration, modulation and/or minimization of at least one clinical symptom related to dysregulation of autophagy.

The term “subject in need thereof” pertains to a subject preferably a mammal, more preferably a human suffering or suspected to be suffering from autophagy dysregulation and ageing disorders.

Furthermore, the term “treatment” refers to alleviation, mitigation, prophylaxis, attenuation, management, regulation, modulation, control, minimization, lessening, decrease, down regulation, up regulation, moderation, inhibition, restoration, suppression, reversal, limitation, blocking prevention, stabilization, amelioration, curing, or healing of autophagy dysregulation.

Notably, the present synergistic composition is non-hazardous, non-toxic, and safe for human consumption without any severe adverse effects. The present composition is also used as preventive therapy/adjuvant therapy/add-on therapy/combination/adjunctive therapy in a subject in need thereof.

Certain compounds of the present invention exist in unsolvated forms as well as solvated forms, including hydrated forms. Further, some compounds of the present invention exist in multiple crystalline or amorphous forms (“polymorphs”). Compounds of the invention are formulated in geometric or, enantiomeric or stereoisomeric forms.

As used herein, the term “pharmaceutically acceptable carriers, diluents or excipients” is purported to mean, without limitation, any adjuvant, carrier, excipient, sweetening agent, diluents, preservative, dye/colorant, flavour enhancer, surfactant, wetting agent, dispersing agent, suspending agent, stabilizer, isotonic agent, solvent, emulsifier, or encapsulating agent, encapsulating polymeric delivery systems or polyethylene glycol matrix, which is acceptable for use in the subject, preferably humans. Excipients also include anti-adherents, antioxidants, binders, coatings, compression aids, disintegrants, dyes (colours), emollients, emulsifiers, fillers (diluents), film formers or coatings, fragrances, glidants (flow enhancers), lubricants, preservatives, sorbents, suspending or dispersing agents, sweeteners, surfactant, anticaking agent, food additives, waters of hydration, or salts.

In another embodiment, the invention relates to a synergistic composition prepared in a manner well known in the pharmaceutical art, and administered by a variety of routes, depending upon whether local or systemic treatment is desired and upon the area to be treated. The preferable route of administration includes but is not limited to sublingual, rectal, topical, parenteral, nasal, or oral.

In yet another embodiment, the present synergistic composition is administered to a subject in need thereof, in the form which is suitable for oral use, such as a tablet, capsule (in the form of delayed release, extended release, sustained release, enteric coated release); hard gelatin capsules, soft gelatin capsules in an oily vehicle, veg capsule, hard or soft cellulose capsule, granulate for sublingual use, effervescent or carbon tablets, aqueous or oily solution, suspension or emulsion, encapsulate, matrix, coat, beadlets, nanoparticles, caplet, granule, particulate, agglomerate, spansule, chewable tablet, lozenge, troche, solution, suspension, rapidly dissolving film, elixir, gel, tablets, pellets, granules, capsules, lozenges, aqueous or oily solutions, suspensions, emulsions, sprays or reconstituted dry powdered form with a liquid medium or syrup; for topical use including transmucosal and transdermal use, such as a cream, ointment, gel, aqueous or oil solution or suspension, salve, parch or plaster; for nasal use, such as a snuff nasal spray or nasal drops; for vaginal or rectal use, such as a suppository; for administration by inhalation, such as a finely divided powder or a liquid aerosol; for sub-lingual or buccal use, such as a tablet, capsule, film, spray. In a further embodiment, the composition is formulated for parenteral use including intravenous, subcutaneous, intramuscular, intravascular, infusion, intraperitoneal, intracerebral, intracerebroventricular, or intradermal routes of administration.

The magnitude of a prophylactic or therapeutic dose typically varies with the nature and severity of the condition to be treated and the route of administration. The dose, and perhaps the dose frequency, will also vary according to the age, body weight and response of the individual patient. In general, the total daily dose (in single or divided doses) ranges from about 1 mg per day to about 5000 mg per day, preferably about 1 mg per day to about 300 mg per day.

In some embodiment, the present invention provides the synergistic composition preserve BBB integrity and reduces the inflammation.

In one preferred embodiment the invention provides a synergistic composition for enhancing TFEB-mediated intracellular clearance comprising therapeutically active exogenous combination of an effective amount N-(3-Aminopropyl)-1,4-butanediamine and N-α-Acetyl-N-β-alanyl-L-histidine and salts thereof which are present in the weight ratio of 1:0.01 to 1:2 along with pharmaceutically acceptable excipients.

In yet another preferred embodiment, the invention provides compositions for enhancing autophagy comprising exogenous blend of therapeutically active N-(3-Aminopropyl)-1,4-butanediamine and N-α-Acetyl-N-β-alanyl-L-histidine present in the weight ratio of 1:0.01 to 1:2 along with pharmaceutically acceptable excipients, wherein the theses two active ingredients act synergistically to improve cell viability.

In one more embodiment, the invention provides synergistic composition comprising N-(3-Aminopropyl)-1,4-butanediamine present in a range of 10% to 95% by weight of the total composition.

In one more embodiment, the invention provides synergistic composition wherein the N-α-Acetyl-N-β-alanyl-L-histidine salt is present in a range of 10% to 85% by weight of the total composition.

In another embodiment, the invention provides synergistic composition useful in the treatment of pregnancy complications.

In another embodiment the invention provides composition wherein the composition is orally administered with effective unit dose of 1 mg to 500 mg.

In some embodiment, the present invention provides synergistic composition for brain specific region comprising specific combination of N-(3-Aminopropyl)-1,4-butanediamine and N-α-Acetyl-N-β-alanyl-L-histidine; wherein N-(3-Aminopropyl)-1,4-butanediamine regulates autophagosomal and lysosomal biogenesis concomitantly N-α-Acetyl-N-β-alanyl-L-histidine acts as mTOR inhibitor that helps in the maintenance of cellular homeostasis by regulating autophagy at the transcriptional level.

The TFEB expression is activated by the synergistic effect wherein N-(3-Aminopropyl)-1,4-butanediamine enhances hypusinated translation factor eIF5A, and N-α-Acetyl-N-β-alanyl-L-histidine inhibits mTORC1 simultaneously, thus promoting efficient shuttling of TFEB from cytosol to the nucleus and transcription of multiple genes implicated in the formation of autophagosomes and lysosomes.

In the context of the present invention, the term “treatment” relates to alleviate, mitigate, prophylaxis, attenuate, manage, regulate, modulate, control, minimize, lessen, decrease, down modulate, up regulate, moderate, inhibit, reverse, restore, suppress, reverse, limit, block, decrease, prevent, inhibit, stabilize, ameliorate or cure, heal autophagy related disorders such as pregnancy complications.

The pregnancy complications include recurrent pregnancy loss, recurrent miscarriage or habitual abortion, abnormal early placentation, embryo pathogenesis, spontaneous abortion, fetal growth restriction, pre-eclampsia, intrauterine growth restriction (IUGR), hormonal imbalance, polycystic ovary syndrome.

Notably, the present synergistic composition is non-hazardous, non-toxic, and safe for human consumption without any adverse effects, therefore the present composition can also be used under preventive therapy/adjuvant therapy/add-on therapy/combination/adjunctive therapy in a subject in need thereof.

Certain compounds of the present invention can exist in unsolvated forms as well as solvated forms, including hydrated forms. Further some compounds of the present invention can exist in multiple crystalline or amorphous forms (“polymorphs”). Compounds of the invention can also exist in geometric or enantiomeric or stereoisomeric forms.

As used herein, the term “pharmaceutically acceptable carriers, diluents or excipients” is purported to mean, without limitation, any adjuvant, carrier, excipient, sweetening agent, diluents, preservative, dye/colorant, flavor enhancer, surfactant, wetting agent, dispersing agent, suspending agent, stabilizer, isotonic agent, solvent, emulsifier, or encapsulating agent, encapsulating polymeric delivery systems or polyethylene glycol matrix which is acceptable for use in the subject, preferably humans. Excipients may also include, for example: antiadherents, antioxidants, binders, coatings, compression aids, disintegrants, dyes (colors), emollients, emulsifiers, fillers (diluents), film formers or coatings, fragrances, glidants (flow enhancers), lubricants, preservatives, sorbents, suspending or dispersing agents, sweeteners, surfactant, anticaking agent, food additives, or waters of hydration, salts.

In another embodiment, the invention relates to synergistic compositions, which can be prepared in a manner well known in the pharmaceutical art, and can be administered by a variety of routes, depending upon whether local or systemic treatment is desired and upon the area to be treated. The preferable route of administration includes but not limited to sublingual, rectal, topical, parenteral, nasal, or oral.

In some embodiment, the present synergistic medicinal composition can be administered to the subject in need thereof, in the form which is suitable for oral use, such as a tablet, capsule (in the form of delayed release, extended release, sustained release, enteric coated release); hard gelatin capsules, soft gelatin capsules in an oily vehicle, veg capsule, hard or soft cellulose capsule, granulate for sublingual use, effervescent or carbon tablets, aqueous or oily solution, suspension or emulsion, encapsulate, matrix, coat, beadlets, nanoparticles, caplet, granule, particulate, agglomerate, spansule, chewable tablet, lozenge, troche, solution, suspension, rapidly dissolving film, elixir, gel, tablets, pellets, granules, capsules, lozenges, aqueous or oily solutions, suspensions, emulsions, sprays or reconstituted dry powdered form with a liquid medium or syrup; for topical use including transmucosal and transdermal use, such as a cream, ointment, gel, aqueous or oil solution or suspension, salve, parch or plaster; for nasal use, such as a snuff nasal spray or nasal drops; for vaginal or rectal use, such as a suppository; for administration by inhalation, such as a finely divided powder or a liquid aerosol; for sub-lingual or buccal use, such as a tablet, capsule, film, spray. Further, the composition can be formulated for parenteral use including intravenous, subcutaneous, intramuscular, intravascular, infusion, intraperitoneal, intracerebral, intracerebroventricular, or intradermal.

Formulations of the present invention suitable for oral administration can be presented as discrete units such as capsules (e.g., soft-gel capsules), cachets or tablets each containing a predetermined amount of the active ingredient; as a powder or granules; as a solution or a suspension in an aqueous liquid or a non-aqueous liquid, syrup; or as an oil-in-water liquid emulsion or a water-in-oil liquid emulsion. The active ingredients can also be presented in the form of a bolus, electuary or paste, bar, energy bars (candy bars), powder, energy drink, ready to drink, granule sachet.

Further, the present composition can be formulated in the form of age-appropriate pediatric oral dosage forms such as syrup, minitablets, chewable formulations, orodispersible films, orodispersible tablets and bioadhesive buccal tablets. It can also be prepared in the form of snack, chocolate bars or other confectionery food products.

In another embodiment, the synergistic composition of the present invention is non-toxic, cost effective, enriched with ingredients, and provides safeguard against problems associated with autophagy impairment without any adverse effect.

In another embodiment of the invention, the diluents are selected from starches, hydrolyzed starches, partially pregelatinized starches, anhydrous lactose, cellulose powder, lactose monohydrate, sugar alcohols such as sorbitol, xylitol and mannitol, silicified microcrystalline cellulose, ammonium alginate, calcium carbonate, calcium lactate, dibasic calcium phosphate (anhydrous/dibasic dehydrate/tribasic), calcium silicate, calcium sulphate, cellulose acetate, corn starch, pregelatinized starch, dextrin, β-cyclodextrin, methylated-β-cyclodextrin, dextrates, dextrose, erythritol, ethyl cellulose, fructose, fumaric acid, glyceryl palmitostearate, magnesium carbonate, magnesium oxide, maltodextrin, maltose, medium-chain triglycerides, polydextrose, polymethacrylates, sodium alginate, sodium chloride, sterilizable maize, sucrose, sugar spheres, talc, trehalose, xylitol, vehicles like petrolatum, dimethyl sulfoxide and mineral oil or the like.

In some embodiment of the invention, the diluent in the composition/formulation is present in a range of 0.1% to 30% by weight of the total composition/formulation.

In yet another embodiment of the invention, the binder is selected from disaccharides such as sucrose, lactose, polysaccharides and their derivatives like starches, cellulose, or modified cellulose such as microcrystalline cellulose and cellulose ethers such as hydroxypropyl cellulose (HPC); hydroxypropyl methyl cellulose (HPMC); sugar alcohols such as xylitol, sorbitol, or mannitol; protein like gelatin; synthetic polymers such as polyvinylpyrrolidone (PVP), polyethylene glycol (PEG), starch, acacia, agar, alginic acid, calcium carbonate, calcium lactate, carbomers, carboxymethylcellulose sodium, carrageenan, cellulose acetate phthalate, chitosan, copovidone, corn starch, pregelatinized starch, cottonseed oil, dextrates, dextrin, dextrose, ethyl cellulose, guar gum, hydrogenated vegetable oil, mineral oil, hydroxyethyl cellulose, hydroxymethyl cellulose, hydroxyl ethyl methyl cellulose, hydroxypropyl cellulose, inulin, cellulose, methyl cellulose, polyvinylpyrrolidone and polyethylene glycol, lactose, liquid glucose, hypromellose, magnesium aluminium silicate, maltodextrin, maltose, methyl-cellulose, microcrystalline cellulose, pectin, poloxamer, polydextrose, polymethacrylates, povidone, sodium alginate, stearic acid, sucrose, sunflower oil, various animal vegetable oils, and white soft paraffin, paraffin, flavorants, colorants and wax.

In further embodiment of the invention, the binder in the composition/formulation is present in a range of 0.1 to 40% by weight of the composition/formulation.

In some embodiment, the antioxidant is selected from tocopherol (vitamin E), sesamol, guaiac resin, methionine, beta-carotene, lycopene, lutein, zeaxanthin, butylated hydroxy anisole (BHA), butylated hydroxytoluene (BHT), sodium ascorbate, sodium metabisulfite (SMB), 1-carnosine, propyl gallate (PG), tertiary butyl hydroquinone, cysteine (CYS), citric acid, tartaric acid, phosphoric acid and ascorbic acid.

In some embodiment of the invention, the amount of antioxidant in the composition/formulation is present in the range of 0.01 to 10% by wt. of the composition/formulation.

In another embodiment of the invention, the lubricant is selected from magnesium stearate, zinc stearate, calcium stearate, glycerin monostearate, glyceryl behenate, glyceryl palmitostearate, hydrogenated castor oil, hydrogenated vegetable oil, light mineral oil, magnesium lauryl sulphate, medium-chain triglycerides, mineral oil, myristic acid, palmitic acid, poloxamer, polyethylene glycol, sodium benzoate, sodium chloride, sodium lauryl sulphate, sodium stearyl fumarate, stearic acid, talc, potassium, or sodium benzoate or the like.

In some embodiment of the invention, the lubricant in the composition/formulation is present in a range of 0.1% to 10.0% by weight of the total composition/formulation.

In another embodiment of the invention, the solubilizing agent is selected from polysorbate 80, sodium lauryl sulphate, anionic emulsifying wax, nonionic emulsifying wax, glyceryl monooleate, phospholipids, polyoxyethylene alkyl ethers, polyoxyethylene castor oil derivatives, polyoxyethylene sorbitan fatty acid esters, polyoxyethylene stearates, polyoxylglycerides, sorbitan esters, triethyl citrate, vitamin E, polyethylene glycol succinate, microcrystalline cellulose, carboxymethylcellulose sodium, diethanolamine, ethylene glycol palmitostearate, glycerin monostearate, hypromellose, hypromellose, acetate succinate, lecithin, polyethylene alkyl ethers, aluminum oxide, poly(methylvinyl ether/maleic anhydride), calcium carbonate, crospovidone, cyclodextrins, fructose, hydroxpropyl betadex, oleyl alcohol, povidone, benzalkonium chloride, benzethonium chloride, benzyl alcohol, benzyl benzoate, cetylpyridinium chloride, inulin, meglumine, poloxamer, pyrrolidone, sodium bicarbonate, starch, stearic acid, sulfobutylether beta cyclodextrin, tricaprylin, triolein, docusate sodium, glycine, alcohol, self-emulsifying glyceryl monooleate, cationic benzethonium chloride, cetrimide, xanthan gum, lauric acid, myristyl alcohol, butylparaben, ethylparaben, methylparaben, propylparaben, sorbic acid or the like.

In another embodiment of the invention, the amount of solubilizing agent or surfactant in the composition/formulation ranges from 0.1% to 10% by weight of the composition/formulation.

In a preferred embodiment of the invention, the solubilizing agent or surfactant is present in a range of 0.1% to 5.0% by weight of the composition/formulation.

In some embodiment of the invention, the glidant is selected from colloidal silicon dioxide, magnesium stearate, fumed silica (colloidal silicon dioxide), starch, talc, calcium phosphate tribasic, cellulose powdered, hydrophobic colloidal silica, magnesium oxide, zinc stearate, magnesium silicate, magnesium trisilicate, silicon dioxide or the like.

In another embodiment of the invention, the glidant in the composition/formulation is present in a range of 0.1% to 5.0% by weight of the total composition/formulation.

In some embodiment of the invention, the stabilizers are selected from the group consisting of alginate, agar, carrageen, gelatin, guar gum, gum arabic, locust bean gum, pectin, starch, xanthan gum, trehalose and likewise.

In some embodiment of the invention, the stabilizer in the composition/formulation is present in a range of 0.1% to 10.0% by weight of the total composition/formulation.

In some embodiment of the invention, the plasticizers are added to coating formulations selected from the group propylene glycol, glycerol, glyceryl triacetate (triacetin), triethyl citrate, acetyl triethyl citrate, diethyl phthalate, acetylated monoglycerides, castor oil, mineral oil and like thereof.

In some embodiment of the invention, the plasticizer in the composition/formulation is present in a range of 0.1% to 5.0% by weight of the total composition/formulation.

In some embodiment of the invention, the solvent is selected from water, alcohol, isopropyl alcohol, propylene glycol, mineral oil, benzyl alcohol, benzyl benzoate, flavored glycol, carbon dioxide, castor oil, corn oil (maize), cottonseed oil, dimethyl ether, albumin, dimethylacetamide, ethyl acetate, ethyl lactate, medium-chain triglycerides, methyl lactate, olive oil, peanut oil, polyethylene glycol, polyoxyl, castor oil, propylene carbonate, pyrrolidone, safflower oil, sesame oil, soybean oil, sunflower oil, water-miscible solvents, organic polar or non-polar solvents or mixtures thereof.

In a preferred embodiment of the invention, the solvent in the composition/formulation is used in a quantity sufficient to make the weight of the composition/formulation 100% by weight.

The additional additives include a polymer, a plasticizer, a sweetener, and a powdered flavor, a preservative, a colorant, a surfactant, and other excipients. The powdered flavor composition includes a flavourant associated with a solid carrier. Coating materials such as synthetic polymers, shellac, corn protein (zein) or other polysaccharides, gelatin, fatty acids, waxes, shellac, plastics, and plant fibers and like thereof are used.

In a preferred embodiment of the invention, the additives are used in a range of 0.1 to 20% w/w of unit dose.

In yet another embodiment, the invention provides the synergistic composition comprising a therapeutic blend of N-(3-Aminopropyl)-1,4-butanediamine and N-α-Acetyl-N-β-alanyl-L-histidine along with pharmaceutical excipients, wherein the pharmaceutical excipients are selected from a diluent, a binder, a lubricant, a glidant, an additive, a surfactant, a stabilizer, or mixtures thereof.

In a preferred embodiment, the invention provides the composition wherein the pharmaceutically acceptable excipients are selected from a group consisting of the diluent is present in a range of 0.1 to 30%; the binder present is present in a range of 0.1 to 25%; the lubricant is present in a range of 0.1 to 10.0%; the glidant is present in a range of 0.1 to 5.0%; the additive is present in a range of 0.1 to 10%; the surfactant is present in a range of 0.1 to 5.0%; the stabilizer is present in a range of 0.1 to 5.0%; %; the antioxidant is present in a range of 0.1 to 5.0%; and the plasticizer is present in a range of 0.1 to 5.0%; by weight of total composition.

In further embodiment compositions containing compounds of the invention, can be conveniently presented in unit dosage form and prepared by any of the methods well known in the art of pharmacy. Preferred unit dosage formulations are those containing an effective dose, or an appropriate fraction thereof, of the active ingredient, or a pharmaceutically acceptable salt thereof.

The magnitude of a prophylactic or therapeutic dose typically varies with the nature and severity of the condition to be treated and the route of administration. The dose, and perhaps the dose frequency, will also vary according to the age, body weight and response of the individual patient.

In general, the total daily dose (in single or divided doses) ranges from about 1 mg per day to about 250 mg per day, preferably about 1 mg per day to about 100 mg per day.

In certain embodiments, the invention provides the potent composition wherein the effective unit dose for an oral administration is formulated in a range of 1 to 100 mg.

It is further recommended that children, patients over 60 years old, initially receive low doses and that the dosage be titrated based on individual physiological responses and/or pharmacokinetics. It can be necessary to use dosages outside these ranges in some cases, as will be apparent to those in the art. The present composition can be used as infant formula as well as adult formula by varying the concentration of active ingredients. Further, it is noted that the dietician or nutritionist or certified physician knows how and when to interrupt, adjust or terminate therapy in conjunction with an individual patient's response.

The use of all examples, or exemplary language (e.g., such as) provided herein, is intended merely to better illuminate the invention, and does not pose a limitation on the scope of the invention unless otherwise claimed.

Various other examples of compositions and modifications or adaptations thereof can be devised by a person skilled in the art after reading the foregoing preferred embodiments without departing from the spirit and scope of the invention. All such further examples, modifications and adaptations are included within the scope of the invention.

It will be appreciated by those versed in the art that the present invention makes available novel and useful nutraceutical compositions and nutraceutical acceptable salts thereof, which have neuroprotective effects in several administration forms. Also, it will be understood by those with knowledge in the dietary supplement and nutraceutical art, that many embodiments of this invention may be made without departing from the spirit and scope of the invention, and the invention is not to be construed as limited, as it embraces all equivalents therein.

The invention may be further illustrated by the following examples, which are for illustrative purposes only and should not be construed as limiting the scope of the invention in anyway.

The present disclosure is therefore to be considered as in all respects illustrative and not restrictive, the scope of the invention being indicated by the appended claims and examples, and all changes or alterations which come within the ambit of equivalency are intended to be encompassed therein.

### EXAMPLES

Having described the basic aspects of the present invention, the following non-limiting examples illustrate specific embodiments thereof. Those skilled in the art will appreciate that many modifications may be made in the invention without changing the essence of invention.

### Example-1

i. Composition 1: Synergistic blend

| Ingredient | w/w % |
|---|---|
| N-(3-Aminopropyl)-1,4-butanediamine | 10% to 95% |
| N-α-Acetyl-N-β-alanyl-L-histidine | 10% to 80% |

ii. Composition 2: Tablet/Capsule

| Ingredient | w/w % unit dose |
|---|---|
| N-(3-Aminopropyl)-1,4-butanediamine | 10 to 95% |
| N-α-Acetyl-N-β-alanyl-L-histidine | 10 to 80% |
| Excipient | 5-20% |
| Average Weight | 100% |

iii. Composition 3: Tablet/Capsule

| Ingredient | w/w % unit dose |
|---|---|
| N-(3-Aminopropyl)-1,4-butanediamine | 10 to 95% |
| N-α-Acetyl-N-β-alanyl-L-histidine | 10 to 80% |
| Diluents | 0.1-10% |
| Binders | 0.1-10% |
| Glidants | 0.1-5% |
| Lubricants | 0.1-5% |
| Stabilizers | 0.1-5% |
| Additives | 0.1-10% |
| Antioxidant | 0.01-5% |
| Solvents | QS |

iv. Composition 4: Tablet/Capsule

| Ingredient | mg per unit dose |
|---|---|
| N-(3-Aminopropyl)-1,4-butanediamine | 3 |
| N-α-Acetyl-N-β-alanyl-L-histidine | 1 |
| Magnesium Stearate | 0.1-5 |
| Hydroxypropyl Methylcellulose | 0.1-5 |
| Microcrystalline Cellulose | 0.1-2 |
| PVP K-30 | 0.1-3 |
| Silicon dioxide | 0.1-1.5 |
| Talc | 0.1-5 |
| Mannitol | 0.1-2 |
| Propylene Glycol | QS |
| Water | QS |
| Average weight | 5-10 mg |

v. Composition 5: Tablet/Capsule

| Ingredient | mg per unit dose |
|---|---|
| N-(3-Aminopropyl)-1,4-butanediamine | 3 |
| N-α-Acetyl-N-β-alanyl-L-histidine | 1 |
| Delta-Tocotrienol | 1 |
| Sodium Benzoate | 0.1-1 |
| Magnesium Stearate | 0.1-2 |
| Ascorbic acid | 0.1-3 |
| Microcrystalline Cellulose | 0.1-2.5 |
| Colloidal Silicon dioxide | 0.1-5 |
| Hydroxypropyl Methylcellulose | 0.1-2 |
| Triethyl citrate | 0.1-3 |
| PVPP | 0.1-2 |
| Talc | 0.1-2 |
| Tween 80 | 0.1-2 |
| Mannitol | 0.1-1 |
| Alcohol | QS |
| Water | QS |
| Average weight | 6-15 mg |

vi. Composition 6: Tablet/Capsule

| Ingredient | mg per unit dose |
|---|---|
| N-(3-Aminopropyl)-1,4-butanediamine | 6 |
| N-α-Acetyl-N-β-alanyl-L-histidine | 1 |
| Butylated hydroxytoluene | 0.1-5 |
| Microcrystalline Cellulose | 0.1-2 |
| Silicon dioxide | 0.1-3 |
| Hydroxypropyl Methylcellulose | 0.1-2.5 |
| Stearic acid | 0.1-3 |
| Dibasic calcium phosphate | 0.1-4 |
| Pregelatinized starch | 0.1-5 |
| Talc | 0.1-2 |
| Tween 80 | 0.1-2 |
| Polydextrose | 0.1-2 |
| PEG | QS |
| Water | QS |
| Average weight | 8-20 mg |

vii. Composition 7: Tablet/Capsule

| Ingredient | mg per unit dose |
|---|---|
| N-(3-Aminopropyl)-1,4-butanediamine | 6 |
| N-α-Acetyl-N-β-alanyl-L-histidine | 1 |
| Delta-Tocotrienol | 2 |
| Microcrystalline Cellulose | 0.1-1 |
| Silicon dioxide | 0.1-2 |
| Butylated hydroxytoluene | 0.1-1 |
| Glycerin | 0.1-2 |
| Ethyl Cellulose | 0.1-2 |
| Hydroxypropyl Methylcellulose | 0.1-2 |
| Magnesium Stearate | 0.1-3 |
| Polyvinylpolypyrrolidone | 0.1-2 |
| Talc | 0.1-5 |
| Polysorbate 20 | 0.1-2 |
| Mannitol | 0.1-2.5 |
| IPA | QS |
| Water | QS |
| Average weight | 10-25 mg |

viii. Composition 8: Tablet/Capsule

| Ingredient | mg per unit dose |
|---|---|
| N-(3-Aminopropyl)-1,4-butanediamine | 3 |
| N-α-Acetyl-N-β-alanyl-L-histidine | 3 |
| Delta-Tocotrienol | 1 |
| Silicon Dioxide | 0.1-2 |
| Medium-chain triglycerides | 0.1-2 |
| Microcrystalline Cellulose | 0.1-3 |
| Dibasic Calcium Phosphate | 0.1-2 |
| Magnesium Stearate | 0.1-2 |
| Croscarmellose sodium | 0.1-2 |
| Polyvinylpyrrolidone | 0.1-2.5 |
| Talc | 0.1-1 |
| Corn Starch | 0.1-1 |
| Sodium ascorbate | 0.1-1 |
| Propylene glycol | 0.1-1 |
| Water | QS |
| Average weight | 8-15 mg |

ix. Composition 9: Tablet/Capsule

| Ingredient | mg per unit dose |
|---|---|
| N-(3-Aminopropyl)-1,4-butanediamine | 1.5 |
| N-α-Acetyl-N-β-alanyl-L-histidine | 1 |
| Microcrystalline Cellulose | 0.1-1 |
| Colloidal silicon dioxide | 0.1-2 |
| Hydroxypropyl Methylcellulose | 0.1-1 |
| Magnesium Stearate | 0.1-2 |
| Polyvinylpyrrolidone | 0.1-2 |
| Calcium Phosphate | 0.1-2 |
| Ascorbic Acid | 0.1-1 |
| Polysorbate 20 | 0.1-2 |
| Talc | 0.1-1 |
| Sucrose | 0.1-1 |
| Mannitol | 0.1-1 |
| Glycerol | 0.1-2 |
| Average weight | 3-10 mg |

x. Composition 10: Tablet/Capsule

| Ingredient | mg per unit dose |
|---|---|
| N-(3-Aminopropyl)-1,4-butanediamine | 1 |
| N-α-Acetyl-N-β-alanyl-L-histidine | 1 |
| Microcrystalline Cellulose | 0.1-1 |
| Silicon dioxide | 0.1-1 |
| Hydroxypropyl Methylcellulose | 0.1-1 |
| Magnesium Stearate | 0.1-2 |
| Zinc Stearate | 0.1-1 |
| Polyvinylpyrrolidone | 0.1-3 |
| Mineral Oil | 0.1-2.5 |
| Sodium benzoate | 0.1-1 |
| Ascorbic Acid | 0.1-2 |
| Polysorbate 20 | 0.1-1 |
| Talc | 0.1-1 |
| Dextrose | 0.1-1 |
| Mannitol | 0.1-1 |
| Water | QS |
| Average weight | 2-10 g |

xi. Composition 11: Tablet/Capsule

| Ingredient | mg per unit dose |
|---|---|
| N-(3-Aminopropyl)-1,4- | 3 |
| butanediamine | |
| N-α-Acetyl-N-β-alanyl-L-histidine | 6 |
| Microcrystalline Cellulose | 0.1-1 |
| Silicon dioxide | 0.1-1 |
| Hydroxypropyl Methylcellulose | 0.1-1 |
| Magnesium Stearate | 0.1-2 |
| Polyvinylpyrrolidone | 0.1-3 |
| Mineral Oil | 0.1-2.5 |
| Sodium benzoate | 0.1-1 |
| Ascorbic Acid | 0.1-2 |
| Polysorbate 20 | 0.1-1 |
| Talc | 0.1-1 |
| Dextrose | 0.1-1 |
| Mannitol | 0.1-1 |
| Water | QS |
| Average weight | 10-20 mg |

### Example 2—Cell Line Study

The purpose of this study is to assess autophagy inducing potential of test substances in Human Monocytes (THP-1) cell line.

The test substance was evaluated for its in vitro potency to induce autophagy in THP-1 cell line. In the given experimental conditions, treatments with the test substance and combination among all the groups tested have shown considerable increase in autophagic activity. (G5-G7) with the combination of two ingredients exhibited the highest percentage of autophagic activity when analysed by CYTO-ID® Autophagy Detection Kit.

### Outline of the Method

The in vitro cytotoxicity was performed on Human Monocytes (THP-1) cell line to find the non-toxic concentrations of the test substances by WST-1 assay and evaluate their autophagic activity in Human Monocytes by CYTO-ID® Autophagy Detection Kit

### Preparation of Test Solution

About 10 mg of the test substances, N-(3-Aminopropyl)-1,4-butanediamine and N-α-Acetyl-N-β-alanyl-L-histidine were dissolved separately with 1 mL RPMI-1640 supplemented with 2% inactivated FBS to obtain a stock solution of 10 mg/mL concentration. Diluted the stock to desired concentrations for carrying out further studies.

### Cell Line and Culture Medium

THP-1 (Human Monocyte cells) was procured from NCCS, India. Stock cells were cultured in RPMI-1640 supplemented with 10% inactivated Fetal Bovine Serum (FBS), penicillin (100 IU/mL), streptomycin (100 μg/mL) and amphotericin B (5 μg/mL) in an humidified atmosphere of 5% CO₂ at 37° C. until confluent. The stock cultures were grown in 25 cm² culture flasks and all experiments were carried out in 96 microtitre plates and 6 well plate (Tarsons India Pvt. Ltd., Kolkata, India).

### Determination of Cell Cytotoxicity by WST-1 Assay

The cell count was adjusted to 100,000 cells/mL using RPMI-1640 containing 10% FBS. To each well of the 96 well micro titre plate, 0.1 mL of the diluted cell suspension was added. After 24 h, 0.1 mL of different test concentrations were added in the micro titre plate. The untreated cells were maintained as cell control for comparison. The plate was then incubated at 37° C. for 24 h in 5% CO₂ atmosphere, and microscopic examination was carried out and observations were noted. After 24 h, 20 μL of WST-1 added to each well and incubated for 3 h at 37° C. in 5% CO₂ atmosphere. The plates were gently shaken and the absorbance was measured using a micro plate reader at a wavelength of 490 nm.

### Study Design

| Groups | Group description | Dose and Treatment |
|---|---|---|
| G1. | Cell Control | No treatment |
| G2. | Reference standard -Rapamycin | Cells were treated with Rapamycin |
| G3. | N-(3-Aminopropyl)-1,4-butanediamine- | Cells were treated with N-(3- |
| | 3 mg | Aminopropyl)-1,4-butanediamine |
| G4. | N-α-Acetyl-N-β-alanyl-L-histidine-1 mg | Cells were treated with N-acetyl-L- |
| | | carnosine |
| G5. | N-(3-Aminopropyl)-1,4-butanediamine + | Cells were treated with the combination |
| | α-Acetyl-N-β-alanyl-L-histidine | of N-(3-Aminopropyl)-1,4- |
| | (3 mg + 0.5 mg) | butanediamine and N-α-Acetyl-N-β- |
| | | alanyl-L-histidine |
| G6. | N-(3-Aminopropyl)-1,4-butanediamine + | Cells were treated with the combination |
| | N-α-Acetyl-N-β-alanyl-L-histidine | of N-(3-Aminopropyl)-1,4- |
| | (3 mg + 1 mg) | butanediamine and N-α-Acetyl-N-β- |
| | | alanyl-L-histidine |
| G7. | N-(3-Aminopropyl)-1,4-butanediamine + | Cells were treated with the combination |
| | N-α-Acetyl-N-β-alanyl-L-histidine | of N-(3-Aminopropyl)-1,4- |
| | (0.1 mg + 5 mg) | butanediamine and N-α-Acetyl-N-β- |
| | | alanyl-L-histidine |

### Sample Treatment

The cell count was adjusted to 1.5-2×10⁵ cells/mL using RPMJ-1640 containing 10% FBS. To each well of the 6 well plates, 2 mL of the diluted cell suspension was added. After 24 h, 1 mL of different test concentrations and combinations of test substances were added. The plate was then incubated at 37° C. for 24 h in a 5% CO₂ atmosphere, and microscopic examination was carried out and observations were noted after 24 h time.

### Estimation of Autophagy Using Flow Cytometer by CYTO-ID

THP-1 cells were incubated with Rapamycin, and test substances in a 6-well plate. Followed by incubation, cells from different groups were harvested and measured using CYTO-ID autophagy detection kit by flow cytometer. All the reagents, standard solutions and samples will be thawed to room temperature before use. The assay was carried out as per manufacturer's instructions.

### Results

TABLE 1
Percentage of cytotoxicity of the test substances in THP-1 cells

| | | | Percentage |
|---|---|---|---|
| | | Concentration | cytotoxicity after |
| Sample code | Sample name | (mg/mL) | treatment |
| G3 | N-(3- | 5 | 39.37 ± 1.37 |
| | Aminopropyl)- | 1 | 22.88 ± 2.80 |
| | 1,4- | 0.5 | 11.79 ± 3.36 |
| | butanediamine | 0.25 | 4.10 ± 1.63 |
| | | 0.12 | 1.20 ± 0.87 |
| | | 0.06 | 0.94 ± 0.18 |
| | | 0.03 | 0.68 ± 2.30 |
| | | 0.01 | 0.74 ± 0.25 |
| | | 0.07 | 0.86 ± 2.08 |
| G4 | N-α-Acetyl-N- | 12.5 | 84.27 ± 1.37 |
| | β-alanyl-L- | 10 | 79.86 ± 0.42 |
| | histidine | 5 | 35.56 ± 1.60 |
| | | 2 | 10.74 ± 1.82 |
| | | 1 | 2.93 ± 4.33 |
| | | 0.5 | 2.62 ± 4.54 |
| | | 0.25 | 0.85 ± 1.45 |
| | | 0.12 | 0.79 ± 1.57 |

TABLE 2
Percentage of THP-1 cells in autophagy analysed by
CYTO-ID ® Autophagy Detection Kit

| | | Percentage cells |
|---|---|---|
| | | expressed |
| Group No. | Group | CYTO-ID |
| G1 | Cell Control (untreated) | 0.02 |
| G2 | Rapamycin | 18.85 |
| G3 | Test -1: N-(3-Aminopropyl)-1,4 | 7.24 |
| | butanediamine- 3 mg | |
| G4 | Test-2: N-α-Acetyl-N-β-alanyl-L- | 4.03 |
| | histidine - 1 mg | |
| G5 | Test 3: N-(3-Aminopropyl)-1,4 | 17.07 |
| | butanediamine + N-α-Acetyl-N-β- | |
| | alanyl-L-histidine (3 mg + 0.5 mg) | |
| G6 | Test 4: N-(3-Aminopropyl)-1,4 | 20.84 |
| | butanediamine + N-α-Acetyl-N-β- | |
| | alanyl-L-histidine (3 mg + 1 mg) | |
| G7 | Test-5: N-(3-Aminopropyl)-1,4 | 48.31 |
| | butanediamine + N-α-Acetyl-N-β- | |
| | alanyl-L-histidine (0.1 mg + 5 mg) | |

### DISCUSSION AND CONCLUSION

In the current study, the effect of test compounds for inducing autophagy in Human Monocytes cell line (THP-1) was analyzed.

Among all the groups tested, combination of test 1 and test 2 in specific ratio show highest percentage of autophagic activity when analysed by CYTO-ID® Autophagy Detection Kit. Most of the test groups and combinations tested have shown considerable increase in autophagic activity. The evidence from this study suggests that the test compound and groups of the test formulations screened could be a good autophagy modulating agent.

From the above results it can be concluded that the combination of test sample found to have good autophagy modulating agent as compared with the individual test sample in a dose dependent manner.

### Abbreviations:

TFEB: Transcription factor EB
mTOR: mechanistic (previously referred to as mammalian) Target of Rapamycin
EIF5A: Eukaryotic translation initiation factor 5A
NAC: N-α-Acetyl-N-β-alanyl-L-histidine
mTORC1: mechanistic (previously referred to as mammalian) Target of Rapamycin
Complex 1
AD: Alzheimer's disease
PD: Parkinsons disease
HD: Huntington's disease
bHLH-Zip: Basic helix-loop-helix leucine zipper
MiT/TFE: microphthalmia/transcription factor E
ICU: Intensive care unit
BBB: Blood-brain barrier
ROS: Reactive oxygen species

1. A composition for enhancing transcription factor EB (TFEB) mediated intracellular clearance, comprising
a. polyamine or its salts, and
b. histidine-containing dipeptide or its salts
along with pharmaceutically acceptable excipients.

2. The composition as claimed in claim 1, wherein said polyamine is N-(3-Aminopropyl)-1,4-butanediamine or its salts thereof.

3. The composition as claimed in claim 1, wherein histidine-containing dipeptide is N-α-Acetyl-N-β-alanyl-L-histidine or its salts thereof.

4. The composition as claimed in claim 1, wherein the N-(3-Aminopropyl)-1,4-butanediamine and the N-α-Acetyl-N-β-alanyl-L-histidine or salts thereof are present in the weight ratio of 1:0.01 to 1:2 along with pharmaceutically acceptable excipients.

5. The composition as claimed in claim 1, wherein the N-(3-Aminopropyl)-1,4-butanediamine salt is present in a range of 10% to 95% by weight of the total composition.

6. The composition as claimed in claim 1, wherein the N-α-Acetyl-N-β-alanyl-L-histidine salt is present in a range of 10% to 80% by weight of the total composition.

7. The composition as claimed in claim 1, wherein the pharmaceutically acceptable excipients are selected from a diluent, a binder, a surfactant, a lubricant, a glidant, an additive, a solvent or mixtures thereof.

8. The composition as claimed in claim 1, wherein the pharmaceutically acceptable excipients are selected from a group consisting of the diluent which is present in a range of 0.1 to 30%; the binder which is present in a range of 0.1 to 25%; the lubricant which is present in a range of 0.1 to 10.0%; the glidant which is present in a range of 0.1 to 5.0%; the additive which is present in a range of 0.1 to 10%; the surfactant which is present in a range of 0.1 to 5.0%; the stabilizer which is present in a range of 0.1 to 5.0%; %; the antioxidant which is present in a range of 0.01 to 5.0%; and the plasticizer which is present in a range of 0.1 to 5.0%; by weight of total composition or mixture thereof.

9. The composition as claimed in claim 1, wherein the composition is useful for the treatment of disorders or diseases related to autophagy, lysosomal biogenesis, mitophagy and lipophagy.

10. The composition as claimed in claim 9, wherein the disorders or diseases are selected from the group consisting of Alzheimer's disease, Parkinson's disease, Huntington's disease, cardiomyopathy, diabetes, liver diseases, muscle diseases, cardiac diseases, cancer, age-related disorders, infection, immunity, autoimmune and inflammatory diseases, pregnancy complications, foetal and infant brain development.

11. The composition as claimed in claim 1, wherein the effective unit dose for an oral administration is formulated in a range of 1 to 500
